(19) United States
(12) Patent Application Publication
(10) Pub. No.: US 2014/0180263 A1
(43) Pub. Date: Jun. 26, 2014

(54) Surgical Instrument

(71) Applicant: Earl C. Downey, (US)

(72) Inventors: Earl C. Downey, Salt Lake City, UT (US); Richard Paul Meyst, El Cajon, CA (US); Kenneth A. Gross, Porter Ranch, CA (US); Stephen Carter, Poway, CA (US); Scott Cameron Thompson, San Diego, CA (US)

(73) Assignee: Earl C. Downey, Salt Lake City, UT (US)

(21) Appl. No.: 13/722,781

(22) Filed: Dec. 20, 2012

(51) Int. Cl. A61B 17/00; F16H 37/12

(52) U.S. Cl. CPC A61B 17/00234; F16H 37/124
USPC 606/1; 29/428; 74/89

(57) ABSTRACT

A surgical instrument is disclosed. The surgical instrument comprises a body operable with a surgical tool and an actuator system supported by the body and configured to convert rotational motion into linear motion to facilitate operation of the surgical tool. The actuator system includes an actuator rod comprising a shaft and a head portion disposed on an end of the shaft. The actuator rod is movable in a translational degree of freedom to facilitate operation of the surgical tool. The actuator system also includes a trigger rotatably supported about the body and moveable by a user. Additionally, the actuator system includes a transition gear coupled to the trigger and comprising a lobe operable to slidably interface with the head portion of the actuator rod. The transition gear is rotatable by the trigger to actuate the actuator rod.

### BACKGROUND

Laparoscopic surgical instruments used for laparoscopic surgery vary significantly in design. Many such surgical instruments are compatible or operable with removable surgical tools that are capable of facilitating both translational and rotational degrees of freedom within a working end, as initiated by the user via the laparoscopic surgical instrument. For example, such surgical tools can facilitate articulation for rotationally positioning the working end, as well as operation or actuation of the working end of the tool, such as positioning and operating a working end in the form of a clamp or cutter.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIG. 1A is an example illustration of a surgical instrument in accordance with an embodiment of the present invention.

FIG. 1B illustrates the surgical instrument of FIG. 1A with exposed interior components.

FIG. 2 is an example illustration of an actuator system of a surgical instrument in accordance with an embodiment of the present invention.

FIG. 3 is an exploded view of the surgical instrument of FIG. 1A showing exposed interior components and an actuator rod of the actuator system in accordance with an embodiment of the present invention.

FIGS. 4A-4C are example illustrations of a transition gear in operation with an actuator rod of an actuator system in accordance with an embodiment of the present invention.

FIGS. 5A-5C are example illustrations of an actuator system of a surgical instrument in operation in accordance with an embodiment of the present invention.

FIG. 6A is an example illustration of an actuator system of a surgical instrument including a chassis in accordance with an embodiment of the present invention.

FIG. 6B is an example illustration of the chassis of FIG. 6A isolated from other components of the actuator system.

FIG. 7 is an example illustration of a body of a surgical instrument configured to interface with the chassis of the actuator system of FIG. 6A in accordance with an embodiment of the present invention.

Reference will now be made to the exemplary embodiments illustrated, and specific language will be used herein to describe the same. It will nevertheless be understood that no limitation of the scope of the invention is thereby intended.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

The phrase “surgical tool,” as used herein, shall be understood to mean any type of instrument, device, system, or assembly that attaches or couples (e.g., that releasably attaches or couples) to a surgical instrument capable of performing a surgical function. Surgical tools will often comprise a working end that can be manipulated by the user to perform an intended function, which manipulation can occur by way of the surgical instrument, the surgical tool, and/or other components operable therewith. Examples of surgical tools include, but are not limited to, scissors, excise tools, scalpels, clamps, mirrors, lasers, lights, cameras, and others.

The phrase “surgical function,” or “surgical procedure,” as used herein, shall be understood to mean any type of activity, action, task, or motion performed by a user by way of a surgical instrument and/or surgical tool coupled thereto. Examples of surgical functions include, but are not limited to, cutting or excising of tissue, clamping or grasping of tissue, and others.

An initial overview of technology embodiments is provided below and then specific technology embodiments are described in further detail later. This initial summary is intended to aid readers in understanding the technology more quickly but is not intended to identify key features or essential features of the technology nor is it intended to limit the scope of the claimed subject matter.

Although many laparoscopic surgical instruments are serviceable, the designs are often complex and include many parts in order to achieve control of a surgical tool to effectuate one or both of translational and rotational movements of the surgical tool and/or a working end operable therewith. Additionally, a surgical instrument having precise operation is typically expensive to produce. Thus, the market would benefit from a less complex surgical instrument design with fewer parts that can be used to operate or manipulate a surgical tool, and/or a precision surgical instrument that is less expensive.

Accordingly, a surgical instrument is disclosed that facilitates translational movement and rotational movement for operating a surgical tool while reducing part count and complexity over previous designs. In one aspect, precision tolerances can be achieved while maintaining low production costs. The surgical instrument can include a body operable with a surgical tool and an actuator system supported by the body and configured to convert rotational motion into linear motion to facilitate operation of the surgical tool. The actuator system can comprise an actuator rod comprising a shaft and a head portion disposed on an end of the shaft. The actuator rod can be movable in a translational degree of freedom to facilitate operation of the surgical tool in a corresponding manner (e.g., actuation of a working end). The actuator system can comprise a trigger rotatably supported about the body and moveable by a user to induce or cause the translational movement. Additionally, the actuator system can comprise a transition gear coupled to the trigger and the actuator rod. The transition gear can include a lobe operable to slidably interface with the head portion of the actuator rod and the transition gear can be rotatable by the trigger to actuate the actuator rod.

In addition, an actuator assembly is disclosed that is operable within a surgical instrument to manipulate a surgical tool coupled to the surgical instrument. The actuator assembly can include a chassis having at least one body interface to facilitate coupling of the chassis to a body of a surgical instrument. In addition, the actuator assembly can include an actuator system supported about the chassis and configured to convert rotational motion into linear motion to facilitate operation of a surgical tool coupled to the surgical instrument.

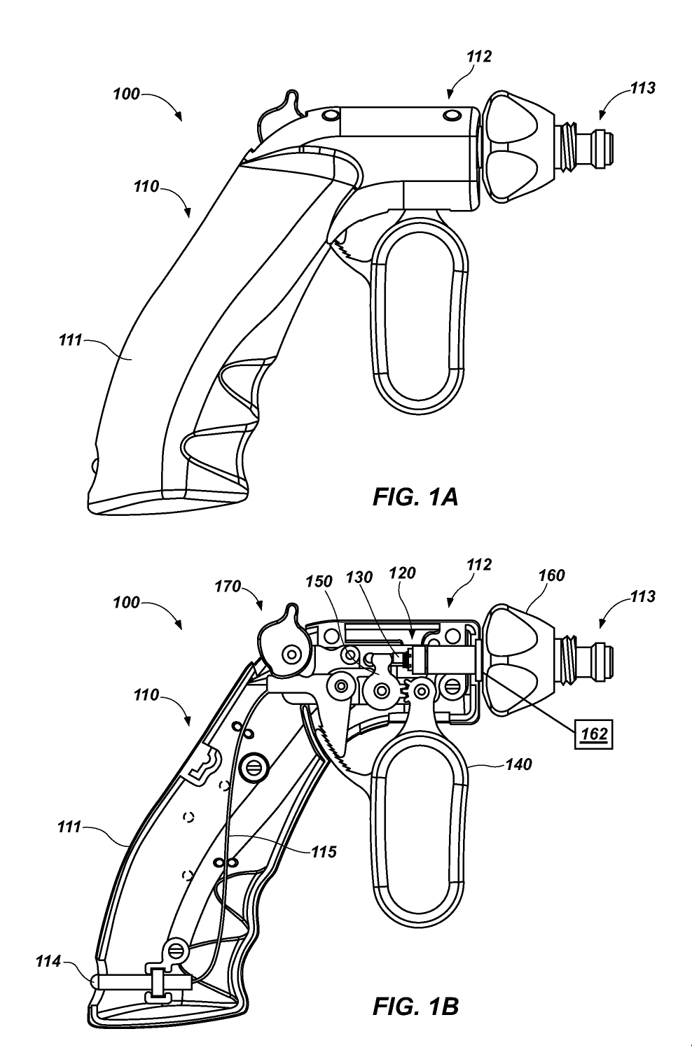

One embodiment of a surgical instrument 100 is illustrated in FIGS. 1A and 1B. The surgical instrument 100 can comprise a body 110 that is operable with a surgical tool (not shown). In terms of functionality, the surgical instrument 100 can be designed to perform the functions or procedures that are most typical in an endoscopic operation, such as cutting tissue, grasping tissue and structures, holding tissues and other objects such as needles, spreading tissues and structures, and so forth. In combination with the body 110 and the mechanisms operable therewith, the surgical tool coupled to the surgical instrument 100 can be capable performing all of these functions, thus making the surgical instrument 100 both versatile and functional. For example, the surgical instrument 100 can provide not only the force required to hold strong tissues, but also control, wherein the user is able to position and control the surgical tool, as well as to sense the amount of pressure being applied.

FIG. 1B illustrates the surgical instrument 100 with an outer cover of the body 110 removed or hidden to reveal additional components and elements of the surgical instrument 100. For example, the surgical instrument 100 can comprise an actuator system 120 supported by the body 110 and configured to convert rotational motion into linear motion to facilitate operation of the surgical tool. As illustrated, the body 110 is configured with a configuration to facilitate operation by the user with a hand of the user in substantially a recognized functional position. The particular configuration of the surgical instrument shown and described herein is not meant to be limiting in any way. Indeed, the surgical instrument, such as the body of the surgical instrument, may be of any suitable configuration.

In the exemplary embodiment shown, the body 110 comprises a handle grip 111 configured to be grasped by a hand of a user, such as a surgeon, and a riser 112 configured to extend a portion of the body 110 away from the hand of the user and to support and removably couple with a surgical tool via a coupling interface 113. A plurality of surgical tools can therefore be interchangeably coupled to the surgical instrument 100 via the coupling interface 113 to enable the operation or function of a given surgical tool with the surgical instrument and the body 110. It should be recognized that the coupling interface 113 can be of any suitable design or configuration that enables coupling of a surgical tool to the surgical instrument 100 and operation of the surgical tool by the surgical instrument 100. Typically, the coupling interface 113 interfaces with a working rod or shaft (not shown) enclosed within a sleeve (not shown) that allows translation and/or rotation of the working rod within the sleeve in order to operate a working end of the surgical tool. As illustrated, the body 110 is specifically configured to orient and maintain the hand of the user in one or more functional positions, as such positions are commonly understood, thus providing a natural and comfortable user interface during use, as well as reducing the possibility of injury to the user, which injuries may include carpal tunnel syndrome, chronic joint stress and others similar in nature.

The handle grip 111 can comprise an ergonomic tubular structure designed to provide significant comfort to the user, as well as to reduce fatigue and other commonly known problems associated with prior related surgical instruments. The handle grip 111 can be further configured as a full hand grip that may be configured to extend beyond or below the user's hand a given distance. By extending the handle grip 111 beyond the hand, the bottom of the handle grip 111 may be set on a steady rest of some sort while performing a surgical function. This is particularly useful in lengthy operations in which a certain surgical function requires precise control for an extended period of time. In one aspect, the handle grip 111 can offset from the riser 112 to provide a body 110 that orients the user's hand within a range of functional positions.

The body 110 may be made of any material or combination of materials common to surgical instruments. Preferably, the body 110 is made of a plastic or lightweight metal material. The body 110 may further comprise some type of gripping texture formed in the handle surface to provide improved grip of the body 110. Alternatively, the body 110 may comprise a rubber or other material gripping element attached or otherwise incorporated into all or a portion of the body 110. In one aspect, the surgical instrument 100 can be adapted for electrocautery. For example, the surgical instrument 100 can include an electrical coupling 114 and a wire 115 to deliver a current to electrically cauterize a surgical site of a patient.

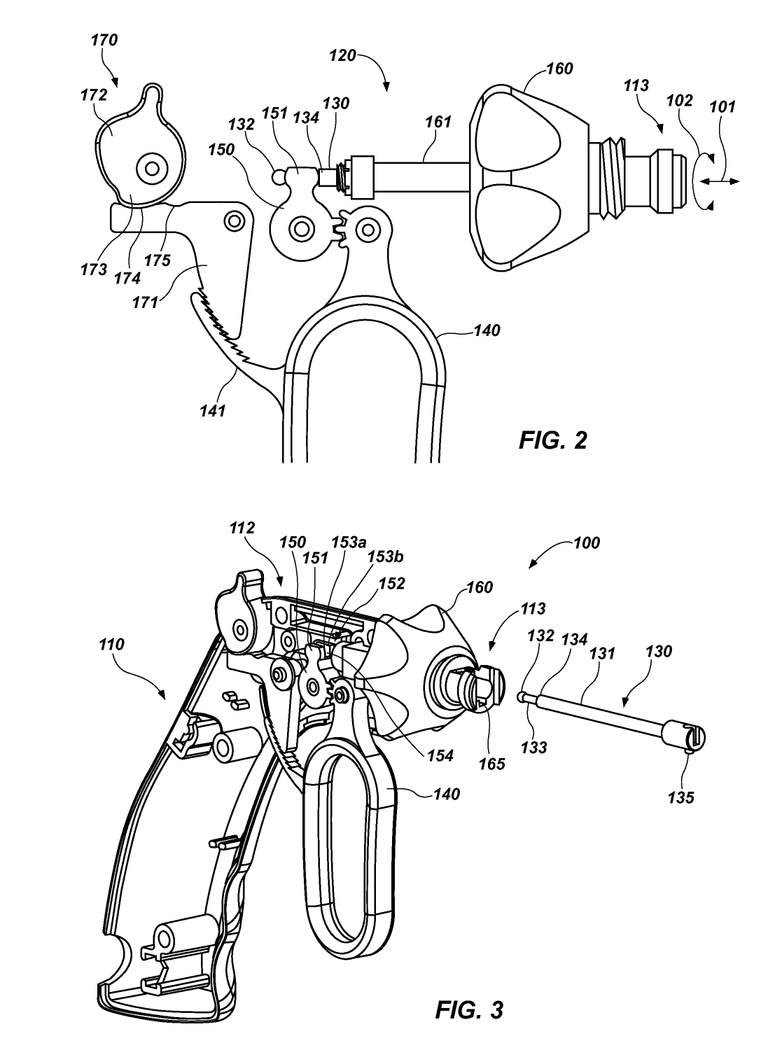

With particular reference to FIGS. 2 and 3, and continued reference to FIGS. 1A and 1B, the actuator system 120 can include an actuator rod 130, a trigger 140, a transition gear 150, and/or a rotator knob 160. In one aspect, the surgical instrument 100 can also include a locking mechanism 170 supported about the body 110. In another aspect, the surgical instrument 100 can include an indexing system 162 operable with the rotator knob 160, which can have a ball biased into a detent to provide discrete rotational positions of the rotator knob 160. For simplicity, the actuator system 120 shown in FIG. 2 is isolated from other components of the surgical instrument 100. In addition, the front perspective exploded view of FIG. 3 illustrates the actuator rod 130 separated from other components to expose certain features of the actuator rod 130.

The trigger 140 and the transition gear 150 can be rotatably or pivotally supported about the body 110. The trigger 140 can be coupled to the transition gear 150, such as by an interface of interlocking gear teeth. The transition gear 150 can include a lobe 151 configured to bear against the actuator rod 130 to cause movement of the actuator rod 130 in a translational degree of freedom 101. The transition gear 150 can therefore be rotatable by the trigger 140 to actuate the actuator rod 130. In one aspect, the transition gear 150, the trigger 140, and/or the actuator rod 130 can be acted on by a biasing force, such as a force provided by a spring, tending to move the respective component toward a predetermined position. For example, a spring associated with the transition gear 150 can be configured to force the transition gear 150 to a neutral position, as discussed hereinafter. In another aspect, the trigger 140 can be configured for interfacing with the user with a two-finger interface, a single-finger interface, a hook, a cushion, or combinations thereof.

The actuator rod 130 can comprise a shaft 131 and a head portion 132 disposed on an end of the shaft 131. In one aspect, the lobe 151 of the transition gear 150 can be operable to slidably interface with the head portion 132 of the actuator rod 130. In another aspect, the shaft 131 of the actuator rod 130 can comprise a reduced cross-sectional area portion 133 defining a shoulder portion 134 that slidably interfaces with a side of the lobe 151 opposite from a side of the lobe 151 that interfaces with the head portion 132 to facilitate bi-directional movement of the actuator rod 130 in the translational degree of freedom 101. In a particular aspect, the lobe 151 of the transition gear 150 can comprise a channel portion 152 formed therein configured to receive the shaft 131 of the actuator rod 130, such as the reduced cross-sectional area portion 133 of the shaft 131. The channel portion 152 can define opposing sides 153a, 153b and a rest 154 formed about the lobe 151 for receiving and/or interfacing with the actuator rod 130.

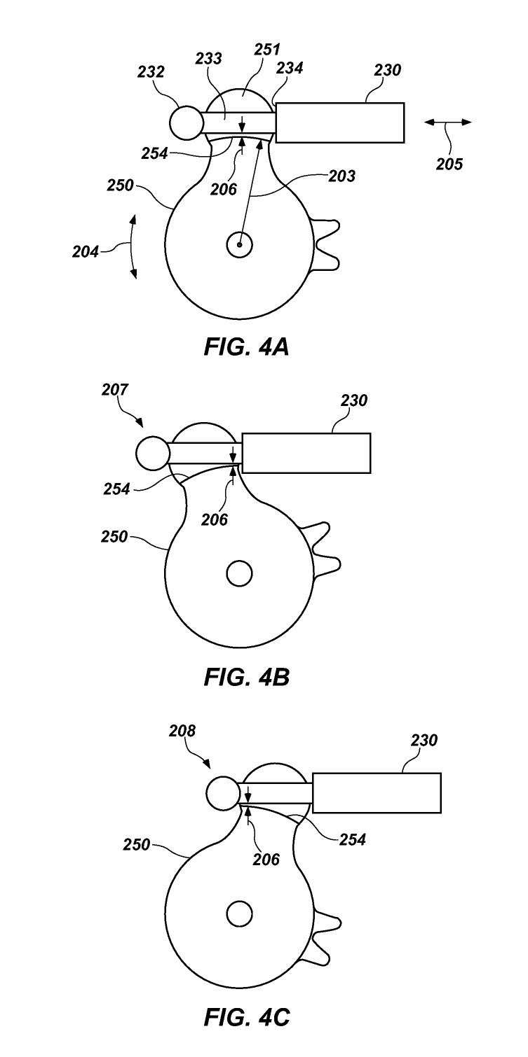

Operation of a transition gear 250 and an actuator rod 230 is illustrated in FIGS. 4A-4C. The transition gear 250 includes a lobe 251 configured to slidably bear against head portion 232 and shoulder portion 234 of the actuator rod 230 as the trigger is actuated and the actuator rod 230 caused to translate in one or more directions, which head portion 232 and shoulder portion 235 are separated by a reduced cross-sectional area portion 233. The transition gear 250 is constrained for rotational movement in direction 204 and the actuator rod 230 is constrained for substantially linear translational movement in direction 205. This configuration therefore facilitates conversion of rotational motion in direction 204 by the transition gear 250 into linear motion of the actuator rod 230 in direction 205. As the transition gear 250 rotates and as the actuator rod 230 moves linearly, the head portion 232 and/or the shoulder portion 234 slide about the edge surfaces of the lobe 251 in order to maintain the linear movement of the actuator rod 230 as the transition gear 250 is rotated.

Due to friction inherent between the sliding interfaces of the lobe 251 and the head and shoulder portions 232, 234, the transition gear 250 can tend to cause the actuator rod 230 to rotate, which may cause bending or deflection of the actuator rod 230 and/or binding or misalignment of the actuator rod 230 with a support constraining the actuator rod 230 to linear movement. In order to limit or eliminate bending and/or binding of the actuator rod 230 and to maintain linear travel or movement, the transition gear 250 can further comprise or include a rest 254. The rest 254 can be configured to contact the shaft, such as the reduced cross-sectional area portion 233, of the actuator rod 230 to provide support for the actuator rod 230 to counteract forces tending to bend and/or bind the actuator rod 230. In one aspect, the rest 254 can comprise an arcuate surface formed on a curve or radius. In a particular aspect, the arcuate surface of the rest 254 can have a radius 203 from the center of rotation of the transition gear 250. Thus configured, a portion of the rest 254 can remain at a predetermined position relative to the reduced cross-sectional area portion 233 of the actuator rod 230 as the transition gear 250 is rotated an the actuator rod 230 linearly displaced. In one aspect, the rest 254 can be configured to be in an offset position 206 relative to the reduced cross-sectional area portion 233 of the actuator rod 230 throughout at least a portion of the range of motion of the transition gear 250, as shown by position 207 in FIG. 4B and by position 208 in FIG. 4C. This can provide support for the actuator rod 230 once the actuator rod 230 has bent or deflected a certain amount sufficient to avoid negative effects due to bending or deflection, while at the same time reducing frictional effects between the reduced cross-sectional area portion 233 and the rest 254. In another aspect, the rest 254 can be configured to be in contact (no offset) with the reduced cross-sectional area portion 233 of the actuator rod 230 throughout at least a portion of the range of motion of the transition gear 250. This can provide constant support for the actuator rod 230. The rest 254 can therefore provide support for the actuator rod 230 as the actuator rod 230 is caused to move in the linear direction 205. The transition gear 250 and the rest 254 can be configured to reduce and/or eliminate friction by facilitating rolling (e.g., no sliding) of the rest 254 about the shaft of the actuator rod 230 as the transition gear 250 is caused to rotate.

Referring again to FIGS. 2 and 3, the actuator rod 130 can also be moveable in a rotational degree of freedom 102 relative to the lobe 151 of the transition gear 150. In one aspect, the rotator knob 160 can be rotatably supported about the body 110 and operable with the actuator rod 130 to cause movement of the actuator rod 130 in a rotational degree of freedom 102 relative to the lobe 151. In other words, the actuator rod 130 can be configured to rotate about the transition gear 150. In a particular aspect, a connector tube 161 can be rotatably supported about the body 110 to couple the rotator knob 160 to the body 110. The connector tube 161 can be configured to slidably receive the actuator rod 130 in the translational degree of freedom 101. The rotator knob 160 can be coupled to the actuator rod 130 in a manner that permits relative motion in the translation degree of freedom 101, while constraining relative motion in the rotational degree of freedom 102.

In this embodiment, the actuator rod 130 can include a protrusion 135 configured to fit within a slot 165 of the coupling interface 113. The slot 165 can allow the protrusion to move in the translational degree of freedom 101 while constraining motion in the rotational degree of freedom 102, such that rotational movement of the rotator knob 160 will also cause rotational movement of the actuator rod 130. A surface of the head 132 and/or shoulder 134 portions of the actuator rod 130 that interface with the lobe 151 of the transition gear 150 can therefore be spherical, conical, parabolic, flat, or combinations thereof that allow for or that facilitate sliding contact between with the lobe 151 in a manner that facilities motion of the actuator rod 130 in the translational 101 and/or rotational 102 degrees of freedom. This can facilitate free rotation of the rotator knob 160 in 360 degrees of the rotational degree of freedom 102 at any given translational position of the actuator rod 130 in the translational degree of freedom 101. The rotator knob 160 can therefore be operable with the actuator rod 130 and configured to facilitate selective rotation of the actuator rod 130 and one or more components of the surgical tool as coupled to the surgical instrument at the coupling interface 113. In addition, the ability of the actuator rod 130 to both translate and rotate due to the interface with the transition gear 150, permits a reduction in parts typically used to facilitate similar translational and rotational degrees of freedom.

In general, the actuator system 120 can be configured to transfer forces to the surgical tool to enable the surgical tool to function as intended. The actuator system 120 can be supported within the riser 112 and the handle of the body 110, thus allowing the user to manipulate the surgical tool at the site of operation by manipulating the body 110 or various components, mechanisms, or systems thereof. In one aspect, the actuator rod 130 can be configured to couple with the working rod associated with the surgical tool at coupling interface 113. For example, the actuator rod 130 can comprise an elongate configuration and be adapted to be substantially in line and coupleable to the working rod at the coupling interface 113. As indicated, the actuator rod 130 can be movable in the translational degree of freedom 101 to facilitate operation or manipulation of the surgical tool. The user can actuate the surgical tool by moving the trigger 140, which acts on the transition gear 150 to cause movement of the actuator rod 130 and a working rod associated with the surgical tool. In addition, the rotator knob 160 can be configured to facilitate selective rotation of the surgical tool and the working end operable therewith, such as by inducing or causing rotation of the sleeve associated with the surgical tool coupled to the surgical instrument 100 via the coupling interface 113.

The surgical instrument further comprises a locking mechanism 170. The locking mechanism 170 can include a ratchet mechanism 171 and a release 172 for the ratchet mechanism 171. The ratchet mechanism 171 can be rotatably supported about the body 110 and configured to engage a ratchet bar 141 of the trigger 140. In one aspect, the ratchet mechanism can be biased, such as by a spring, toward the ratchet bar 141 for automatic engagement of the ratchet mechanism 171 with the ratchet bar 141. The release 172 can be configured to disengage the ratchet mechanism 171 from the ratchet bar 141, such as by acting against the biasing force of the spring. In one aspect, the release 172 can include a cam 173 configured to ride in detents 174, 175 configured to facilitate selective engagement and disengagement of the ratchet mechanism 171 and the ratchet bar 141.

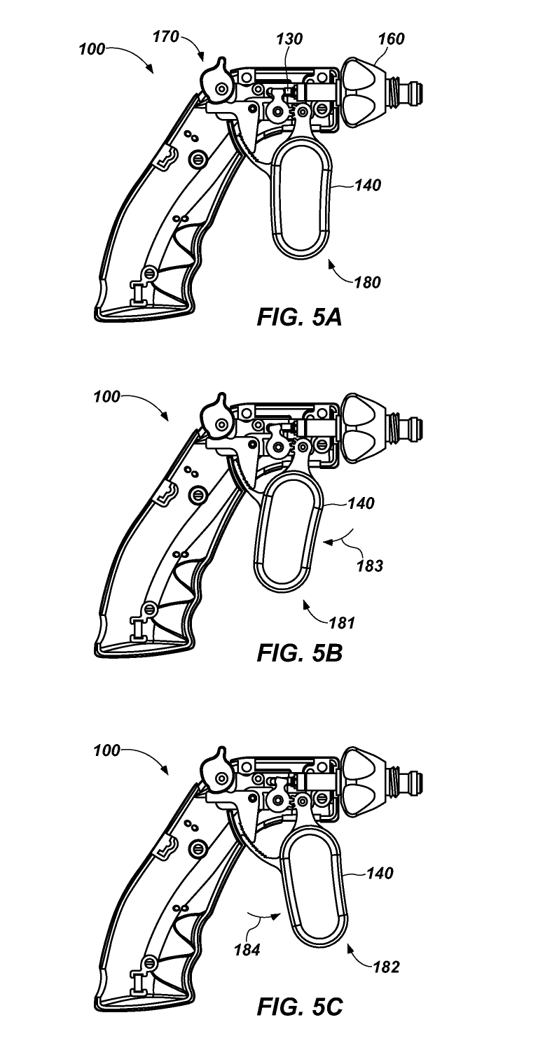

FIGS. 5A-5C illustrate the surgical instrument 100 in operation. For example, FIG. 5A illustrates the surgical instrument 100 in a neutral position, somewhere between the translational limits of actuator rod 130. The user can elect to move the trigger 140 in either direction 183 or 184 shown in FIGS. 5B 5C, respectively, depending on the desired operation of the surgical tool corresponding with a retracted position 181 or an extended position 182 of the trigger 140. It should be noted that the rotator knob 160 can be rotated at any time with the actuator rod 130 in any translational position to operate the surgical tool. In addition, the locking mechanism 170 can be operated to engage or disengage the trigger 140 as desired to lock or permit movement of the trigger 140 for controlling the surgical tool.

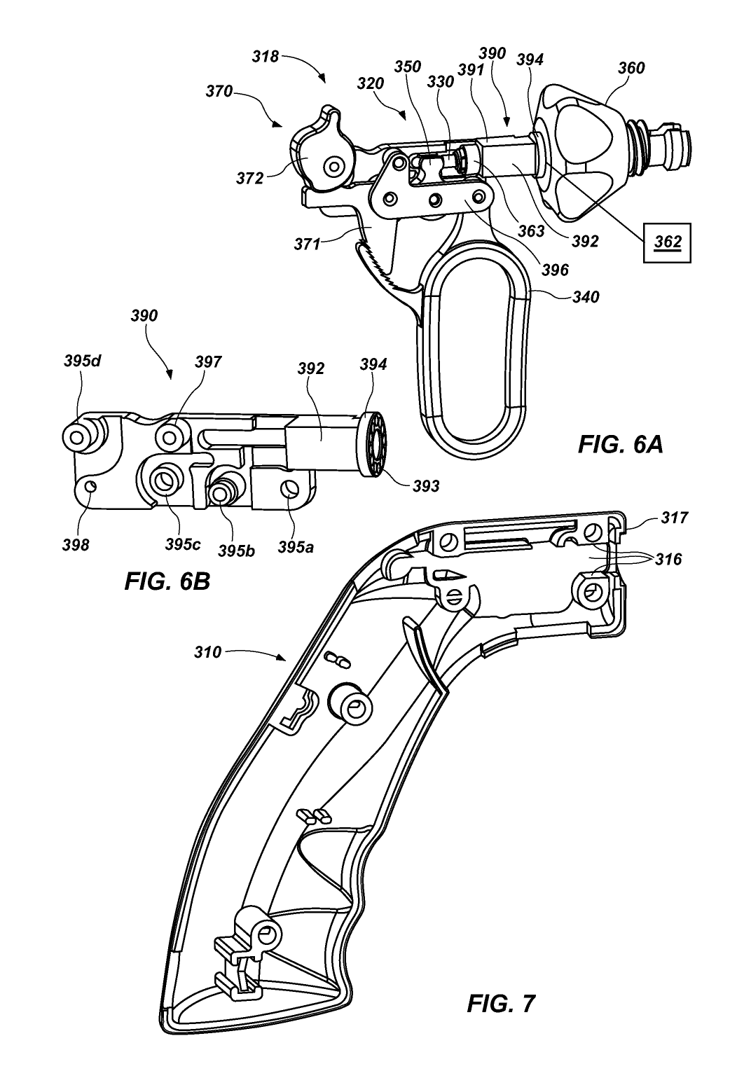

With reference to FIGS. 6A-7, illustrated is an actuator assembly 318 in accordance with an exemplary embodiment. In this embodiment, the actuator assembly, including an actuator system, can be coupled or coupleable to a body 310 of a surgical instrument and operable within the surgical instrument to manipulate a surgical tool coupled to the surgical instrument similarly as described above. The actuator assembly 318 can further comprise or include a chassis 390 comprising at least one body interface 391 to facilitate coupling of the chassis 390 to the body 310 of the surgical instrument, wherein the chassis (and anything coupled thereto) are supported by the body 310 of the surgical instrument.

The actuator assembly 318 can further comprise or include an actuator system 320 supported about the chassis 390, wherein the actuator system 320 can be configured to convert rotational motion into linear motion to facilitate operation of a surgical tool coupled to the surgical instrument, similarly as discussed above. Thus, the actuator assembly 318 can comprise a self-contained assembly that includes the chassis 390 and all of the actuator system 320 components in an integrated structure that operates within, but independent of the body 310, although supported about or by the body via the chassis 390. In other words, the chassis 390 can serve as the foundation or support structure for the various components of the actuator system 320, independent of the body of the surgical instrument. The actuator system 320 components can be mounted directly to the chassis 390 rather than being mounted directly to the body of the surgical instrument.

This can be advantageous when it is desirable to reduce material and manufacturing costs while maintaining precision operation of the actuator system 320. For example, a low cost material, such as a plastic, and low cost manufacturing process, such as injection molding, can be used to manufacture the body 310. Such a material and process, however, may not provide sufficiently high tolerances desired in the embodiments where the actuator system components are coupled directly to the body of the surgical instrument. Specifically, it may be difficult to achieve sufficiently high tolerances upon locating and supporting the actuator system components directly about the body. The chassis 390, however, can be made of a material, such as a high-grade metal, that can be manufactured to tight tolerances for locating and supporting the actuator system components. By supporting the components of the actuator system 320 about the chassis 390, high or tight tolerances can be achieved and maintained over time and through repeated use of the surgical instrument. As such, providing a chassis 390 that interfaces with and is supported about the body, and that itself directly supports the actuator system components, can greatly improve both initial quality, durability and performance of the surgical instrument. Instead of locating and directly supporting the actuator system components, the base 310 of the surgical instrument can serve primarily as an interface for the user's hand and as a support for the chassis 390, while also functioning to at least partially cover the actuator system 320 components.

In one aspect, body interfaces of the chassis 390, such as body interface 391, can comprise a flat surface to provide a simple interface with one or more chassis interfaces 316 formed in or about the body 310. The chassis interfaces 316 can comprise any suitable feature or structure to interface with the body interface 391 of the chassis 390, such as a recess, platform, etc. The body 310 can comprise at least two components that combine to capture or secure the chassis 390 within the body 310, such as by “sandwiching” the chassis 390 and securing the chassis 390 without fasteners. The chassis 390 can be secured to the body with one or more fasteners.

In other embodiments, the chassis 390 can be configured to be removably or permanently coupled to the body 310. Configuring the chassis 390 to be separable or removable from the body and the surgical instrument can facilitate interchangeability of the chassis, and the actuation system supported thereon, with another similarly configured surgical instrument. In other words, it may be desirable to replace a surgical instrument. By providing a removable chassis and associated actuation system, the chassis and the associated actuation system can be removed from the existing surgical instrument and inserted into the new surgical instrument. Or, if replacement is not necessary, providing a removable chassis and associated actuation system can facilitate cleaning and/or sterilization of the surgical instrument. Indeed, by being able to access the chassis and the actuation system and temporarily remove it, cleaning of the surgical instrument and its component parts can be enhanced and improved.

Furthermore, the body 310 can comprise at least two components that are separable from one another to facilitate access of the chassis 390 and the actuator system 320. This can be useful for disassembly and maintenance of the actuator system 320, as well as for cleaning and/or sterilization of the surgical instrument, including its interior and interior components.

It should be recognized that the chassis 390 can itself be configured in a variety of ways to support any form or configuration or type of actuator system. In particular, it should be recognized that the chassis 390 can support any actuator system component or other suitable component, such as those disclosed herein. As shown, the chassis 390 can be configured to support, or have mounted thereon or thereto, an actuator rod, a transition gear, a trigger, a rotator knob, a locking mechanism, an indexing system, etc.

For example, the chassis 390 can comprise a capture portion 392 for receiving and supporting the actuator rod 330. In one aspect, the capture portion 392 can be configured to interface with and rotatably support a connector tube, as described hereinabove, to couple the rotator knob 360 to the chassis 390. A fastener 363, such as a nut, can be used to secure the connector tube and, therefore the rotator knob 360, to the chassis 390. Accordingly, the capture portion 392 of the chassis 390 can also be configured to interface with the fastener 363 in a manner that facilitates rotation of the fastener 363 as well as the connector tube. In addition, the chassis 390 can include one or more features 393 to function with an indexing system 362 operable with the rotator knob 360 to provide discrete rotational positions of the rotator knob 360. For example, the features 393 can comprise detents or recesses configured to interface with a ball biased into one of the detents. As shown, the chassis 390 can further be configured such that at least a portion 394 of the chassis 390 is configured to extend outside a front portion 317 of the body 310 of the surgical instrument to provide support for and interface with a rotator knob 360. In one aspect, the portion 394 can be configured to include the features 393 to function with the indexing system 362 for the rotator knob 360.

With particular reference to FIG. 6B, the chassis 390 can include one or more interface features 395a-d configured to interface with and support the trigger 340, the transition gear 350, and/or components of a locking mechanism 370, such as a ratchet mechanism 371 and a release 372 for the ratchet mechanism 371 when supported by the chassis 390. One or more of the interface features 395a-d can comprise a protrusion, such as a pin 395b, to be received within an opening of a mating component. Additionally, one or more of the interface features 395a-d can comprise an opening, such as hole 395a, to receive a protrusion of a mating component. In one aspect, the actuation assembly 318 can further comprise a cover plate 396 configured to facilitate securing the actuator rod 330, the trigger 340, the transition gear 350, and/or components of the locking mechanism 370 to the chassis 390. Accordingly, the chassis 390 can also be configured to couple with and support the cover plate 396 with a dedicated cover plate interface feature 397 and/or by utilizing interface features 395a-d. The cover plate 396 can further function to interface with the body of the surgical instrument, and to help support the chassis 390 within or about the body of the surgical instrument. In another aspect, the chassis 390 can include interface feature 398 configured to interface and couple with a wire for delivering a current to electrically cauterize a surgical site of a patient. The interface feature 398 can be configured to facilitate soldering or any other technique for electrically coupling the wire to the chassis 390. It should be recognized that the chassis 390 can include any feature, such as a channel, groove, protrusion, hole, etc., that may facilitate interfacing, supporting, or otherwise accommodating any actuator assembly 318 component or related surgical instrument component.

In accordance with one embodiment of the present invention, a method of facilitating construction of a surgical instrument is disclosed. The method can comprise facilitating support of an actuator system about a chassis. The method can further comprise facilitating coupling of the chassis to a body of a surgical instrument, wherein the actuator system is configured to convert rotational motion into linear motion to facilitate operation of a surgical tool coupled to the surgical instrument. In one aspect of the method, facilitating coupling of the chassis to a body of the surgical instrument can comprise facilitating coupling of at least one body interface of the chassis to the body of the surgical instrument. In another aspect of the method, the actuator system can comprise an actuator rod to be supported about the chassis, and comprising a shaft and a head portion disposed on an end of the shaft, the actuator rod movable in a translational degree of freedom to facilitate operation of the surgical tool. The actuator system can also comprise a trigger to be rotatably supported about the chassis, and moveable by a user. In addition, the actuator system can comprise a transition gear to be supported about the chassis, and coupled to the trigger, the trigger comprising a lobe operable to slidably interface with the head portion of the actuator rod, the transition gear being rotatable by the trigger to actuate the actuator rod. It is noted that no specific order is required in this method, though generally in one embodiment, these method steps can be carried out sequentially.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

What is claimed is:

1. A surgical instrument, comprising:
a body operable with a surgical tool; and
an actuator system supported by the body and configured to convert rotational motion into linear motion to facilitate operation of the surgical tool, the actuator system comprising:
an actuator rod comprising a shaft and a head portion disposed on an end of the shaft, the actuator rod movable in a translational degree of freedom to facilitate operation of the surgical tool;
a trigger rotatably supported about the body, and moveable by a user; and
a transition gear coupled to the trigger, and comprising a lobe operable to slidably interface with the head portion of the actuator rod, the transition gear being rotatable by the trigger to actuate the actuator rod.

2. The surgical instrument of claim 1, wherein the actuator rod is moveable in a rotational degree of freedom relative to the lobe of the transition gear.

3. The surgical instrument of claim 1, further comprising a rotator knob rotatably supported about the body, the rotator knob being operable with the actuator rod, wherein the actuator rod is moveable in a rotational degree of freedom relative to the lobe of the transition gear upon rotation of the rotator knob.

4. The surgical instrument of claim 3, further comprising a connector tube rotatably supported about the body, the connector tube coupling the rotator knob to the body, and configured to slidably receive the actuator rod in the translational degree of freedom.

5. The surgical instrument of claim 1, wherein the shaft of the actuator rod comprises a reduced cross-sectional area defining a shoulder that slidably interfaces with a side of the lobe opposite from the head portion, to facilitate bi-directional movement of the actuator rod.

6. The surgical instrument of claim 1, wherein the lobe of the transition gear further comprises a channel portion formed therein configured to receive the shaft, the channel portion defining opposing sides and a rest for the actuator rod.

7. The surgical instrument of claim 6, wherein the rest comprises an arcuate surface configured to limit at least one of deflection and misalignment of the actuator rod.

8. The surgical instrument of claim 6, wherein the actuator rod is supported in an offset position relative to the rest.

9. The surgical instrument of claim 1, further comprising a chassis supported about the body, wherein the actuator rod, the trigger and the transition gear of the actuator system are mounted to the chassis.

10. The surgical instrument of claim 9, wherein the chassis is made of metal.

11. The surgical instrument of claim 9, wherein the chassis and the actuator system supported by the chassis are removable from the body.

12. The surgical instrument of claim 9, further comprising a locking mechanism supported about the chassis, the locking mechanism comprising a ratchet mechanism and a release for the ratchet mechanism.

13. The surgical instrument of claim 9, wherein the body comprises a chassis interface configured to interface with a body interface on the chassis.

14. The surgical instrument of claim 9, wherein at least a portion of the chassis extends outside a front portion of the body of the surgical instrument to interface with a rotator knob.

15. The surgical instrument of claim 9, wherein the body comprises at least two components separable from one another to facilitate accessibility of the chassis and the actuator system.

16. An actuator assembly operable within a surgical instrument to manipulate a surgical tool coupled to the surgical instrument, the actuator assembly comprising:
a chassis comprising at least one body interface to facilitate coupling of the chassis to a body of a surgical instrument; and
an actuator system supported about the chassis and configured to convert rotational motion into linear motion to facilitate operation of a surgical tool coupled to the surgical instrument.

17. The actuator assembly of claim 16, wherein the chassis is removably coupled to the body.

18. The actuator assembly of claim 16, wherein the chassis is made of metal.

19. The actuator assembly of claim 16, wherein the chassis further comprises an indexing system operable with a rotator knob, the indexing system having a ball biased into a detent to provide discrete rotational positions of the rotator knob.

20. The actuator assembly of claim 16, wherein the actuator system comprises:
an actuator rod supported about the chassis, and comprising a shaft and a head portion disposed on an end of the shaft, the actuator rod movable in a translational degree of freedom to facilitate operation of a surgical tool;
a trigger rotatably supported about the chassis, and moveable by a user; and
a transition gear supported about the chassis, and coupled to the trigger, the trigger comprising a lobe operable to slidably interface with the head portion of the actuator rod, the transition gear being rotatable by the trigger to actuate the actuator rod.

21. The actuator assembly of claim 20, further comprising a cover plate configured to facilitate securing of the actuator rod, the trigger, and the transition gear to the chassis.

22. A method of facilitating construction of a surgical instrument, comprising:
facilitating support of an actuator system about a chassis; and
facilitating coupling of the chassis to a body of a surgical instrument, wherein the actuator system is configured to convert rotational motion into linear motion to facilitate operation of a surgical tool coupled to the surgical instrument.

23. The method of claim 22, wherein facilitating coupling of the chassis to a body of the surgical instrument comprises facilitating coupling of at least one body interface of the chassis to the body of the surgical instrument.

24. The method of claim 22, wherein the actuator system comprises:
an actuator rod to be supported about the chassis, and comprising a shaft and a head portion disposed on an end of the shaft, the actuator rod movable in a translational degree of freedom to facilitate operation of the surgical tool;
a trigger to be rotatably supported about the chassis, and moveable by a user; and
a transition gear to be supported about the chassis, and coupled to the trigger, the trigger comprising a lobe operable to slidably interface with the head portion of the actuator rod, the transition gear being rotatable by the trigger to actuate the actuator rod.
